(19) United States
(12) Patent Application Publication
(10) Pub. No.: US 2025/0161562 A1
(43) Pub. Date: May 22, 2025

(54) MULTI-LUMEN INTRAVENOUS TUBING SYSTEMS

(71) Applicant: CareFusion 303, Inc., San Diego, CA (US)

(72) Inventor: Ryan Callahan, Long Beach, CA (US)

(21) Appl. No.: 18/513,454

(22) Filed: Nov. 17, 2023

(51) Int. Cl. A61M 5/168; A61M 39/10

(52) U.S. Cl. CPC A61M 5/168; A61M 39/10

(57) ABSTRACT

Multiple lumen intravenous tubing systems can include multi-lumen tubing that can be coupled with a manifold, where the multi-lumen tubing includes multiple lumens extending within the tubing and which are fluidly coupled with counterpart fluid flow paths of the manifold when the multi-lumen tubing is connected to an outlet of the manifold, and where the manifold includes more than one inlet and an outlet with each inlet fluidly coupled to a respective fluid flow paths that can be fluidically separated from the other fluid flow paths of the manifold to permit directing a first fluid from through the manifold and multi-lumen tubing, and a second fluid through the manifold and multi-lumen tubing, without the first and second fluids intersecting.

### BACKGROUND

The present disclosure relates generally to intravenous (IV) fluid delivery devices and systems for conveying more than one medical fluid to a patient. More specifically, the present disclosure relates to multi-lumen IV tubing systems that can include a manifold and multi-lumen tubing for directing more than one medical fluid to a patient while maintaining separation of each medical fluid along the multi-lumen tubing.

In the field of infusion therapy, one or more medicament can be directed to a patient using an IV set, which can include a medicament source, IV tubing, and a medical connector to fluidly couple the IV set with the patient. For example, the medicament source can be a fluid container which may include saline and/or a medication, and the medical connector can be a needleless connector, a y-site connector, and/or a catheter.

The description provided in the background section should not be assumed to be prior art merely because it is mentioned in or associated with the background section. The background section may include information that describes one or more aspects of the subject technology.

### SUMMARY

In some instances of infusion therapy, there is a need to deliver multiple medications to the patient, such that an IV extension set having multiple branches of tubing through which the multiple medications may be dispensed to the patient is used. For example, a main infusion line may be intravenously coupled to a patient and to multiple branches of tubing, and any of the multiple branches of tubing may be utilized by one or more medical professional to deliver one or more medicament to the patient, such as by an anesthesiologist and other doctor.

When intravenously delivering a medicament to a patient, one or more portion of the IV set may be primed before injecting a medication and may be flushed after injecting the medication in order to increase the likelihood that an intended volume of medication is delivered to the patient within the desire time. In some instances, a medical professional must consider the length and volume of the IV set and the tendency of the medication to adhere to or move more slowly against an inner surface of the IV set when determining whether a medication is delivered to the patient as intended. In other instances, a medical professional must position themselves very close to a patient to directly inject a medication into a patient's catheter, potentially displacing or disturbing other medical professionals attending to the patient.

A multi-lumen IV tubing system of the present disclosure can be implemented to ensure that an intended volume of medication is delivered to the patient within the desire time, to provide a more efficient system that can be configured to receive and direct more than one medicament to a patient without the need for multiple separate IV tubing connections, without requiring injecting the medicament directly into a catheter port, and without requiring the flushing of a medicament using a bolus or continuous infusion of saline. The multi-lumen IV tubing system of the present disclosure can include a manifold and a multi-lumen tubing connected to the manifold, where the manifold can have more than one inlet port and the multi-lumen tubing can include fluidly separated lumens, and where each inlet port of the manifold is fluidly coupled to a lumen of the multi-lumen tubing.

Accordingly, in some embodiments, multi-lumen intravenous tubing systems are provided that can comprise a multi-lumen tubing comprising a primary lumen and a secondary lumen, the primary lumen and the secondary lumen each extending from a first end of the multi-lumen tubing to a second end of the multi-lumen tubing, and the secondary lumen is fluidly separated from the primary lumen, and a manifold comprising a first fluid flow path, a second fluid flow path, a first inlet, a second inlet, and an outlet, the first fluid flow path extending from the first inlet to the outlet, and the second fluid flow path extending from the second inlet to the outlet, wherein the first fluid flow path is fluidly separated from the second fluid flow path, wherein the first end of the multi-lumen tubing is coupled to the outlet of the manifold with the first fluid flow path fluidly coupled to the primary lumen, and the second fluid flow path fluidly coupled to the secondary lumen.

Some embodiments, multi-lumen intravenous tubing systems include a medicament source, a fluid connector, a manifold, and a multi-lumen tubing, the manifold comprising a first fluid flow path that extends from a first inlet to an outlet of the manifold, a second fluid flow path that extends from a second inlet to the outlet, wherein the first inlet is fluidly coupled to the medicament source, and the multi-lumen tubing comprising a first end, a second end, a primary lumen, and a secondary lumen, the first end coupled to the outlet of the manifold, the second end coupled to the fluid connector, and each of the primary lumen and the secondary lumen extending from a first end to the second end of the multi-lumen tubing, and wherein a first fluid can move from the medicament source, through the first fluid flow path of the manifold and the primary lumen, to the fluid connector, and wherein a second fluid can move from the second fluid flow path, through the second fluid flow path and the secondary lumen, to the fluid connector.

In some instances, the present disclosure provides methods for providing a multi-lumen intravenous tubing system, the method comprising providing a manifold comprising a first fluid flow path that extends from a first inlet to an outlet of the manifold, a second fluid flow path that extends from a second inlet to the outlet of the manifold, and providing a multi-lumen tubing comprising a first end coupled to the outlet of the manifold such that a primary lumen of the multi-lumen tubing is fluidly coupled with only the first fluid flow path, and a secondary lumen of the multi-lumen tubing is fluidly coupled with only the second fluid flow path.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The following figures are included to illustrate certain aspects of the embodiments and should not be viewed as exclusive embodiments. The subject matter disclosed is capable of considerable modifications, alterations, combinations, and equivalents in form and function, as will occur to those skilled in the art and having the benefit of this disclosure.

FIG. 1 illustrates a multi-lumen IV tubing system in use with a patient, in accordance with aspects of the present disclosure.

FIG. 2 illustrates a perspective view of multi-lumen tubing, in accordance with aspects of the present disclosure.

FIG. 3 illustrates a perspective view of another embodiment of multi-lumen tubing, in accordance with aspects of the present disclosure.

FIG. 4 illustrates a perspective view of another embodiment of multi-lumen tubing, in accordance with aspects of the present disclosure.

FIG. 5 illustrates a top plan view of a manifold, in accordance with aspects of the present disclosure.

FIG. 6 illustrates a front elevation view of the manifold of FIG. 5, in accordance in accordance with aspects of the present disclosure.

FIG. 7 illustrates a top plan view of another embodiment of a manifold, in accordance with aspects of the present disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions may be provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

In accordance with some embodiments, the present application discloses various features and advantages of multi-lumen IV tubing systems. The multi-lumen IV tubing systems of the present disclosure can provide for efficient and safe delivery of medicaments to a patient. The multi-lumen IV tubing systems provide a single multi-lumen tubing and manifold that can be configured to receive and direct more than one medicament to a patient while resisting mixing of the medicaments along the multi-lumen tubing. The multi-lumen IV tubing systems can resist mixing of the medicaments by providing more than one separate fluid flow path from the manifold and through the multi-lumen tubing. The more than one fluid flow path of the multi-lumen IV tubing system can merge at an end of the multi-lumen tubing that is coupled to a patient, such as at a catheter connection, for example. In some aspects, the multi-lumen IV tubing systems provide rapid or immediate delivery of a medicament to a patient, relative to the time required to deliver a medicament that is injected into a fluid flow path of saline in a main line of an IV set.

Additionally, in some embodiments of the present disclosure, a first inlet of the manifold can be coupled to a flushing fluid, such as saline, and a second inlet of the manifold can be used to inject a medication into the multi-lumen IV tubing system, wherein the fluid directed through the first inlet (e.g., flushing fluid) and the fluid directed through the second inlet (e.g., medication) are separated through the manifold and the multi-lumen tubing. Thus, the flushing fluid coupled to the first inlet of the manifold can be used to flush or clear the medical connector (e.g., catheter) at the end of the multi-lumen tubing, thereby preventing unintended mixing of medicaments.

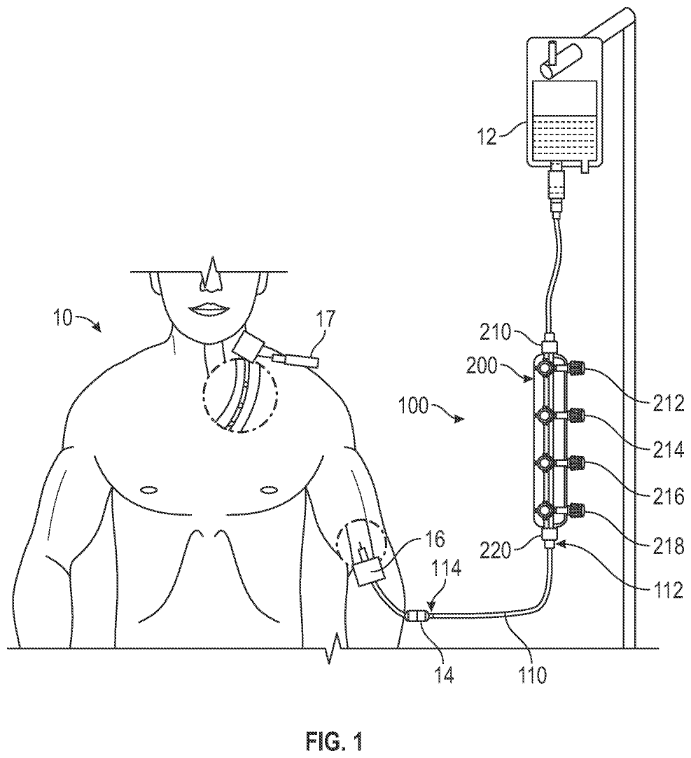

An example of the multi-lumen IV tubing system in use with a patient is illustrated in FIG. 1. The multi-lumen IV tubing system 100 is coupled to a portion of an IV set and is configured to direct one or more medicament to a patient 10. The multi-lumen IV tubing system 100 can include multi-lumen tubing 110 and a manifold 200. In the illustrated example, a first inlet 210 of the manifold is fluidly coupled to a medicament bag 12, and an outlet 220 of the manifold is fluidly coupled to the multi-lumen tubing 110.

The multi-lumen tubing 110 includes a first end 112 and a second end 114. The first end 112 of the multi-lumen tubing is coupled to the outlet 220 of the manifold, and the second end 114 of the multi-lumen tubing is coupled to a medical connector 14 at or near the patient 10. The medical connector 14 can be a device coupled to the patient for infusion therapy. For example, the medical connector 14 can be a catheter 16 itself or can be coupled to a catheter 16, such that the one or more medicament directed through the multi-lumen IV tubing system 100 can enter into a blood vessel of the patient 10 through the catheter.

Although the first inlet 210 of the manifold is coupled to a medicament bag 12 in the example shown in FIG. 1, it should be understood that the present disclosure contemplates embodiments in which the manifold is not coupled to a medicament bag, and or in which a medicament can be directly injected into the manifold using one or more inlet of the manifold.

To permit injecting one or more medicament into the multi-lumen IV tubing system, the manifold includes more than one inlet, such as a second inlet 212, a third inlet 214, a fourth inlet 216, and a fifth inlet 218. In another embodiment of the present disclosure, the manifold can comprise more or less than five inlets.

The manifold 200 includes a separate fluid flow path from each of the more than one inlet to the outlet. For example, the manifold 200 includes a separate fluid flow path from each of the inlets 210, 212, 214, 216, 218 to the outlet 220. At the outlet 220, each fluid flow path is coupled to a lumen of the multi-lumen tubing such that the separate fluid flow paths are maintained from the manifold 200 to the multi-lumen tubing 110, and along the multi-lumen tubing 110.

To maintain separate fluid flow paths, the multi-lumen tubing includes more than one lumen, where each of the lumens extend from the first end 112 to the second end 114 of the multi-lumen tubing.

The lumens extend along the multi-lumen tubing and each lumen forms a cross-sectional profile. The cross-sectional profile of each lumen is defined transverse to a longitudinal axis between the first end 112 and the second end 114 of the multi-lumen tubing. Further, each lumen defines or forms a profile shape. It should be understood that the present disclosure contemplates embodiments in which each of the lumens have the same profile shape and in which at least one lumen has a different profile shape than other lumens. It is also contemplated that the lumens can be oriented between the first end 112 and the second end 114 such that a longitudinal axis of each lumen is parallel relative to each other.

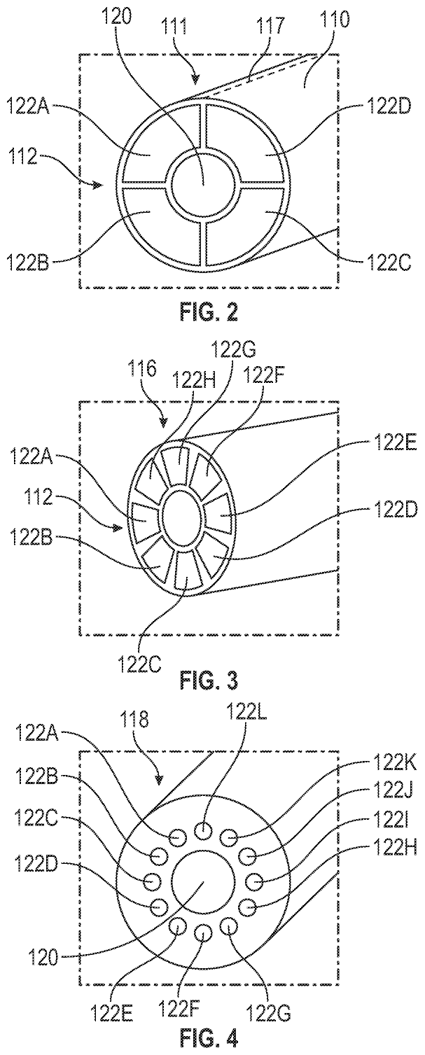

Referring to FIG. 2, a perspective view showing a first end 112 of an embodiment of the multi-lumen tubing 111 is illustrated having a primary lumen 120 and more than one secondary lumen 122A, 122B, 122C, 122D. The multi-lumen tubing 111 can have any number of secondary lumens and can have a quantity of primary and secondary lumens that corresponds with a quantity of inlets in the manifold 200. For example, the multi-lumen tubing 111 includes five lumens, which can include the primary lumen 120 and secondary lumens 122A, 122B, 122C, 122D.

Each of the primary lumen 120 and the secondary lumens 122A, 122B, 122C, 122D extend from the first end 112 to the second end 114 of the multi-lumen tubing 111, and each of the primary lumen 120 and the secondary lumens 122A, 122B, 122C, 122D are fluidly separated from each other to resist mixing between fluids that are directed through each of the lumens. When an end of the multi-lumen tubing, e.g., the first end 112, is coupled to the outlet 220 of the manifold, each of the primary lumen 120 and the secondary lumens 122A, 122B, 122C, 122D are fluidly coupled to a separate fluid flow path of the manifold.

The multi-lumen tubing 111 can be configured with the secondary lumens 122A, 122B, 122C, 122D positioned around the primary lumen 120 such that a longitudinal primary axis, defined by the primary lumen 120, is parallel or approximately parallel to a longitudinal secondary axis, defined by each of the secondary lumens 122A, 122B, 122C, 122D. In some embodiments of the present disclosure, the secondary lumens 122A, 122B, 122C, 122D are positioned concentrically around the primary lumen 120. In any embodiments of the present disclosure, the position, orientation, and configuration of the lumens of the multi-lumen tubing can correspond with and align with the position, orientation, and configuration of the fluid flow paths at the outlet 220 of the manifold.

In some embodiments of the present disclosure, the multi-lumen tubing 111 can include an orientation feature 117 that is configured to provide a reference for coupling the multi-lumen tubing with the manifold such that each of the lumens of the multi-lumen tubing are fluidly coupled with a fluid flow path of the manifold. The orientation feature 117, for example, can be visually observed, such as a line along the tubing or a symbol. The orientation feature 117 can also be a structure or feature that is tactile or identifiable by touching the orientation feature 117. For example, the orientation feature 117 can be a surface of the multi-lumen tubing that is flattened, relative to the adjacent portions of the outer surface of the multi-lumen tubing. In another example, the orientation feature 117 can be any of the first or second end 112, 114 of the multi-lumen tubing that forms a plane that is less than or greater than ninety degrees relative to the longitudinal axis of the multi-lumen tubing. In another example, the orientation feature 117 can be a notch or step formed in the end of the first end 112 and/or second end 114 of the multi-lumen tubing.

Each of the primary and secondary lumens also define a volume that can correspond with an intended flow rate for each lumen. The volume of each lumen can be determined by the respective lumen profile area relative to the length of the tubing. In embodiments in which each of the lumen has the same profile area, the volume for each lumen will be approximately equal. In some embodiments, each of the lumens can have a volume that is approximately equal when one or more lumen has a different profile shape than the profile shape of the other lumens, when the profile area of each lumen is approximately equal. Thus, when the profile area of each lumen is equal, the multi-lumen tubing can have the same volume per unit length of the multi-lumen tubing.

Referring to the embodiment of the multi-lumen tubing 111 illustrated in FIG. 2, the primary lumen has a circular profile shape and the secondary lumens 122A, 122B, 122C, 122D have an irregular or sector-shaped profile formed between the primary lumen and the outer surface of the multi-lumen tubing. The area of each of the secondary lumens 122A, 122B, 122C, 122D is greater than an area of the primary lumen 120 in some embodiments of the multi-lumen tubing.

Turning now to FIG. 3, another embodiment of the multi-lumen tubing 116 is illustrated having a primary lumen 120 and eight secondary lumens 122A, 122B, 122C, 122D, 122E, 122F, 122G, 122H. Although the primary lumen 120 has a different shape profile than the secondary lumens 122A, 122B, 122C, 122D, 122E, 122F, 122G, 122H, the profile area of each lumen is approximately equal.

FIG. 4 illustrates another embodiment of a multi-lumen tubing 118 having a primary lumen and secondary lumens each having a circular profile shape. Although the present disclosure contemplates embodiments in which the multi-lumen tubing can have any number of primary lumens and secondary lumens, the embodiments of the multi-lumen tubing 118 is illustrated having twelve secondary lumens 122A, 122B, 122C, 122D, 122E, 122F, 122G, 122H, 122I, 122J, 122K, 122L positioned concentrically around the primary lumen 120.

The multi-lumen tubing 110 of the present disclosure can couple with an outlet of a manifold 200 such that each fluid flow path of the manifold is fluidly coupled with a lumen of the multi-lumen tubing 110. The present disclosure contemplates that any embodiment of the manifold includes more than one inlet and an outlet, where a fluid flow path extends from each inlet to the outlet without intersecting each other.

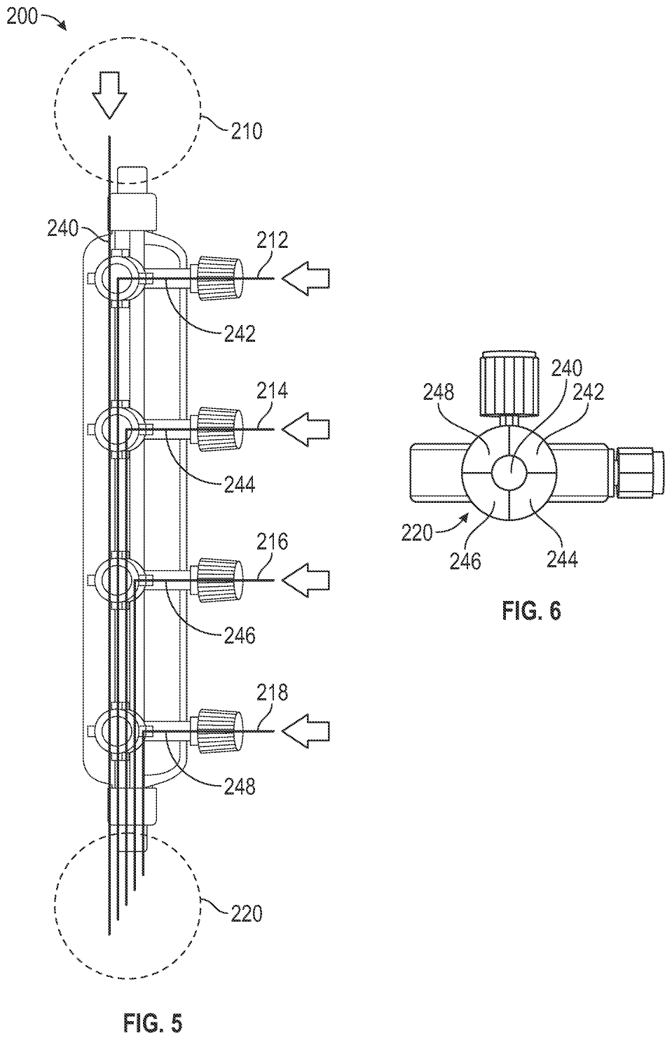

Referring to FIG. 5, an embodiment of the manifold 200 is illustrated with fluid flow paths of the manifold illustrated as lines overlaying the manifold for clarity and ease of reference in this disclosure. The manifold 200 includes a first inlet 210, a second inlet 212, a third inlet 214, a fourth inlet 216, a fifth inlet 218, and an outlet 220. A first fluid flow path 240 extends from the first inlet 210 to the outlet 220, a second fluid flow path 242 extends from the second inlet 212 to the outlet 220, a third fluid flow path 244 extends from the third inlet 214 to the outlet 220, a fourth fluid flow path 246 extends from the fourth inlet 216 to the outlet 220, a fifth fluid flow path 248 extends from the fifth inlet 218 to the outlet 220.

At the outlet 220, illustrated in FIG. 6, each of the fluid flow paths 240, 242, 244, 246, 248 are positioned adjacent to one another to fluidly couple with a corresponding lumen of the multi-lumen tubing, such as the multi-lumen tubing 111 illustrated in FIG. 2.

In some embodiments of the present disclosure, the manifold can be configured with a first fluid flow path that is fluidly coupled to the outlet and to a second fluid flow path to permit at least a portion of a fluid moving through the first fluid flow path to move into the second fluid flow path. By permitting at least a portion of a fluid moving through the first fluid flow path to move into the second fluid flow path of the manifold, the second fluid flow path can be flushed by the fluid from the first fluid flow path.

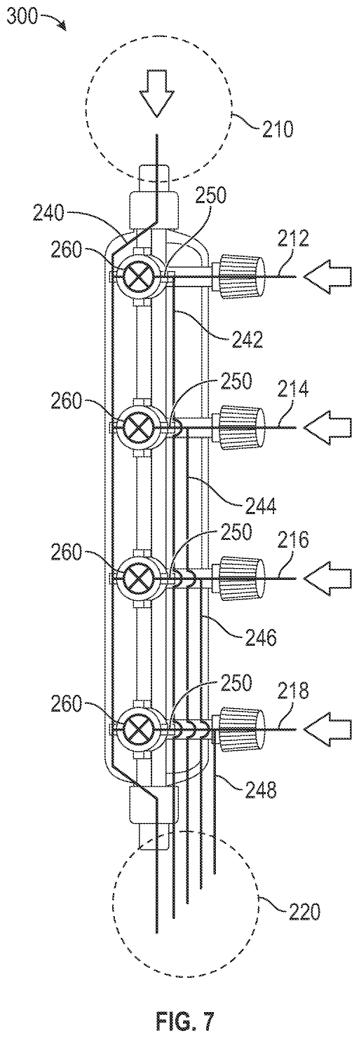

An embodiment of a manifold 300 that is configured to selectively permit at least a portion of a fluid moving through the first fluid flow path to move into the second fluid flow path is illustrated in FIG. 7. Features of the manifold 300 that are similar to the manifold 200, are identified with the same reference numbers for clarity and brevity of the disclosure. The manifold 300 includes a first inlet 210, a second inlet 212, a third inlet 214, a fourth inlet 216, a fifth inlet 218, and an outlet 220. A first fluid flow path 240 extends from the first inlet 210 to the outlet 220, a second fluid flow path 242 extends from the second inlet 212 to the outlet 220, a third fluid flow path 244 extends from the third inlet 214 to the outlet 220, a fourth fluid flow path 246 extends from the fourth inlet 216 to the outlet 220, a fifth fluid flow path 248 extends from the fifth inlet 218 to the outlet 220.

Additionally, a tertiary fluid flow path 250 extends from the first fluid flow path 240 to each of the second through fifth flow paths 242, 244, 246, 248. The tertiary fluid flow path 250 permits at least a portion of a fluid moving through the first fluid flow path 140 to move into the respective second through fifth flow paths 242, 244, 246, 248 connected thereto. By fluidly coupling the first fluid flow path 240 to another fluid flow path using the tertiary fluid flow path 250, a fluid can be directed through the first fluid flow path 240 to flush and/or prime any of the respective second through fifth flow paths 242, 244, 246, 248 connected thereto.

In some embodiments, one or more tertiary fluid flow path 250 can also include a valve 260 that can selectively permit or resist movement of a fluid between the first fluid flow path 240 and the respective second through fifth flow paths 242, 244, 246, 248 connected thereto.

The present disclosure contemplates that the first inlet 210 can be used to inject a bolus of medicament into the manifold 300 to flush or prime a fluid flow path, and it is also contemplated that the first inlet 210 can be coupled to medicament source, such as an IV bag, to provide a constant stream of medicament through the manifold and the tertiary fluid path 250.

In any embodiment of the present disclosure, the manifold 200 and multi-lumen tubing 110 permit the injection of more than one medicament directly to the patient using a single multi-lumen tubing extending from the manifold to the patient. Thus, features of the present disclosure provide for rapid, efficient, and safe delivery of medicaments directly, from an inlet of a manifold and through a lumen of a multi-lumen tubing that is connected directly to the inlet, to a patient.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 5. The other clauses can be presented in a similar manner.

Clause 1. A multi-lumen intravenous tubing system comprising a multi-lumen tubing comprising a primary lumen and a secondary lumen, the primary lumen and the secondary lumen each extending from a first end of the multi-lumen tubing to a second end of the multi-lumen tubing, and the secondary lumen is fluidly separated from the primary lumen; and a manifold comprising a first fluid flow path, a second fluid flow path, a first inlet, a second inlet, and an outlet, the first fluid flow path extending from the first inlet to the outlet, and the second fluid flow path extending from the second inlet to the outlet, wherein the first fluid flow path is fluidly separated from the second fluid flow path; wherein the first end of the multi-lumen tubing is coupled to the outlet of the manifold with the first fluid flow path fluidly coupled to the primary lumen, and the second fluid flow path fluidly coupled to the secondary lumen.

Clause 2. The multi-lumen intravenous tubing system of Clause 1, wherein the primary lumen forms a primary lumen cross-sectional profile that is transverse to a longitudinal primary axis of the primary lumen, and the secondary lumen forms a secondary lumen cross-sectional profile that is transverse to a longitudinal secondary axis of the secondary lumen, and wherein an area of the primary lumen cross-sectional profile is greater than an area of the secondary lumen cross-sectional profile.

Clause 3. The multi-lumen intravenous tubing system of any of the preceding clauses, wherein the primary lumen forms a first volume between the first end and the second end of the multi-lumen tubing, and the secondary lumen forms a second volume between the first end and the second end of the multi-lumen tubing, and wherein the first volume is greater than the second volume.

Clause 4. The multi-lumen intravenous tubing system of any of the preceding clauses, wherein the primary lumen defines a longitudinal primary axis, and the secondary lumen defines a longitudinal secondary axis, and wherein the longitudinal primary axis is parallel with the longitudinal secondary axis.

Clause 5. The multi-lumen intravenous tubing system of any of the preceding clauses, wherein the secondary lumen comprises a first secondary lumen and a second secondary lumen.

Clause 6. The multi-lumen intravenous tubing system of Clause 5, wherein each of the first secondary lumen and the second secondary lumen are positioned concentrically around the primary lumen.

Clause 7. The multi-lumen intravenous tubing system of Clause 6, wherein each of the first secondary lumen and the second secondary lumen define a longitudinal secondary axis that is parallel to a longitudinal primary axis of the primary lumen.

Clause 8. The multi-lumen intravenous tubing system of any of the preceding clauses, wherein the primary lumen forms a primary lumen cross-sectional profile having a circular shape, and the secondary lumen forms a secondary lumen cross-sectional profile having a circular shape.

Clause 9. The multi-lumen intravenous tubing system of any of the preceding clauses, wherein a length of the primary lumen, from the first end to the second end of the multi-lumen tubing, is equal to a length of the secondary lumen, from the first end to the second end of the multi-lumen tubing.

Clause 10. The multi-lumen intravenous tubing system of any of the preceding clauses, wherein the multi-lumen tubing comprises an outer surface forming a cross-sectional profile of multi-lumen tubing, and wherein the cross-sectional profile of multi-lumen tubing is circular.

Clause 11. The multi-lumen intravenous tubing system of any of the preceding clauses, wherein the manifold comprises a tertiary fluid flow path that extends from the first fluid flow path to the second fluid flow path.

Clause 12. The multi-lumen intravenous tubing system of Clause 11, wherein the manifold comprises a valve configured to selectively permit a fluid to move from the first fluid flow path to the second fluid flow path.

Clause 13. A multi-lumen intravenous tubing system comprising: a medicament source; a fluid connector; a manifold comprising a first fluid flow path that extends from a first inlet to an outlet of the manifold, a second fluid flow path that extends from a second inlet to the outlet, wherein the first inlet is fluidly coupled to the medicament source; and a multi-lumen tubing comprising a first end, a second end, a primary lumen, and a secondary lumen, the first end coupled to the outlet of the manifold, the second end coupled to the fluid connector, and each of the primary lumen and the secondary lumen extending from a first end to the second end of the multi-lumen tubing; wherein a first fluid can move from the medicament source, through the first fluid flow path of the manifold and the primary lumen, to the fluid connector, and wherein a second fluid can move from the second fluid flow path, through the second fluid flow path and the secondary lumen, to the fluid connector.

Clause 14. The multi-lumen intravenous tubing system of Clause 13, wherein the secondary lumen is fluidly separated from the primary lumen.

Clause 15. The multi-lumen intravenous tubing system of any of Clauses 13 and 14, wherein the manifold comprises a tertiary fluid flow path that extends from the first fluid flow path to the second fluid flow path.

Clause 16. The multi-lumen intravenous tubing system of Clause 15, wherein the manifold comprises a valve configured to selectively permit at least a portion of the first fluid to move from the first fluid flow path to the second fluid flow path.

Clause 17. The multi-lumen intravenous tubing system of any of Clauses 13 to 16, wherein the primary lumen forms a first volume between the first end and the second end of the multi-lumen tubing, and the secondary lumen forms a second volume between the first end and the second end of the multi-lumen tubing, and wherein the first volume is greater than the second volume.

Clause 18. A method for providing a multi-lumen intravenous tubing system, the method comprising: providing a manifold comprising a first fluid flow path that extends from a first inlet to an outlet of the manifold, a second fluid flow path that extends from a second inlet to the outlet of the manifold; and providing a multi-lumen tubing comprising a first end coupled to the outlet of the manifold such that a primary lumen of the multi-lumen tubing is fluidly coupled with only the first fluid flow path, and a secondary lumen of the multi-lumen tubing is fluidly coupled with only the second fluid flow path.

Clause 19. The method of Clause 18, wherein providing a manifold further comprises providing a tertiary fluid flow path between the first fluid flow path to the second fluid flow path, such that at least a portion of a fluid can move from the first fluid flow path to the second fluid flow path.

Clause 20. The method of Clause 19, further comprising providing a valve along the tertiary fluid flow path to selectively permit or resist movement of the fluid between the first fluid flow path and the second fluid flow path.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, or operations in the processes or methods disclosed are illustrations of exemplary approaches. Based upon implementation preferences or scenarios, it is understood that the specific order or hierarchy of steps, operations or processes may be rearranged. Some of the steps, operations or processes may be performed simultaneously. In some implementation preferences or scenarios, certain operations may or may not be performed. Some or all of the steps, operations, or processes may be performed automatically, without the intervention of a user. The accompanying method claims present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but are to be accorded the full scope consistent with the language of the claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A multi-lumen intravenous tubing system comprising:
a multi-lumen tubing comprising a primary lumen and a secondary lumen, the primary lumen and the secondary lumen each extending from a first end of the multi-lumen tubing to a second end of the multi-lumen tubing, and the secondary lumen is fluidly separated from the primary lumen; and
a manifold comprising a first fluid flow path, a second fluid flow path, a first inlet, a second inlet, and an outlet, the first fluid flow path extending from the first inlet to the outlet, and the second fluid flow path extending from the second inlet to the outlet, wherein the first fluid flow path is fluidly separated from the second fluid flow path;
wherein the first end of the multi-lumen tubing is coupled to the outlet of the manifold with the first fluid flow path fluidly coupled to the primary lumen, and the second fluid flow path fluidly coupled to the secondary lumen.

2. The multi-lumen intravenous tubing system of claim 1, wherein the primary lumen forms a primary lumen cross-sectional profile that is transverse to a longitudinal primary axis of the primary lumen, and the secondary lumen forms a secondary lumen cross-sectional profile that is transverse to a longitudinal secondary axis of the secondary lumen, and wherein an area of the primary lumen cross-sectional profile is greater than an area of the secondary lumen cross-sectional profile.

3. The multi-lumen intravenous tubing system of claim 1, wherein the primary lumen forms a first volume between the first end and the second end of the multi-lumen tubing, and the secondary lumen forms a second volume between the first end and the second end of the multi-lumen tubing, and wherein the first volume is greater than the second volume.

4. The multi-lumen intravenous tubing system of claim 1, wherein the primary lumen defines a longitudinal primary axis, and the secondary lumen defines a longitudinal secondary axis, and wherein the longitudinal primary axis is parallel with the longitudinal secondary axis.

5. The multi-lumen intravenous tubing system of claim 1, wherein the secondary lumen comprises a first secondary lumen and a second secondary lumen.

6. The multi-lumen intravenous tubing system of claim 5, wherein each of the first secondary lumen and the second secondary lumen are positioned concentrically around the primary lumen.

7. The multi-lumen intravenous tubing system of claim 6, wherein each of the first secondary lumen and the second secondary lumen define a longitudinal secondary axis that is parallel to a longitudinal primary axis of the primary lumen.

8. The multi-lumen intravenous tubing system of claim 1, wherein the primary lumen forms a primary lumen cross-sectional profile having a circular shape, and the secondary lumen forms a secondary lumen cross-sectional profile having a circular shape.

9. The multi-lumen intravenous tubing system of claim 1, wherein a length of the primary lumen, from the first end to the second end of the multi-lumen tubing, is equal to a length of the secondary lumen, from the first end to the second end of the multi-lumen tubing.

10. The multi-lumen intravenous tubing system of claim 1, wherein the multi-lumen tubing comprises an outer surface forming a cross-sectional profile of multi-lumen tubing, and wherein the cross-sectional profile of multi-lumen tubing is circular.

11. The multi-lumen intravenous tubing system of claim 1, wherein the manifold comprises a tertiary fluid flow path that extends from the first fluid flow path to the second fluid flow path.

12. The multi-lumen intravenous tubing system of claim 11, wherein the manifold comprises a valve configured to selectively permit a fluid to move from the first fluid flow path to the second fluid flow path.

13. A multi-lumen intravenous tubing system comprising:
a medicament source;
a fluid connector;
a manifold comprising a first fluid flow path that extends from a first inlet to an outlet of the manifold, a second fluid flow path that extends from a second inlet to the outlet, wherein the first inlet is fluidly coupled to the medicament source; and
a multi-lumen tubing comprising a first end, a second end, a primary lumen, and a secondary lumen, the first end coupled to the outlet of the manifold, the second end coupled to the fluid connector, and each of the primary lumen and the secondary lumen extending from a first end to the second end of the multi-lumen tubing;
wherein a first fluid can move from the medicament source, through the first fluid flow path of the manifold and the primary lumen, to the fluid connector, and wherein a second fluid can move from the second fluid flow path, through the second fluid flow path and the secondary lumen, to the fluid connector.

14. The multi-lumen intravenous tubing system of claim 13, wherein the secondary lumen is fluidly separated from the primary lumen.

15. The multi-lumen intravenous tubing system of claim 13, wherein the manifold comprises a tertiary fluid flow path that extends from the first fluid flow path to the second fluid flow path.

16. The multi-lumen intravenous tubing system of claim 15, wherein the manifold comprises a valve configured to selectively permit at least a portion of the first fluid to move from the first fluid flow path to the second fluid flow path.

17. The multi-lumen intravenous tubing system of claim 13, wherein the primary lumen forms a first volume between the first end and the second end of the multi-lumen tubing, and the secondary lumen forms a second volume between the first end and the second end of the multi-lumen tubing, and wherein the first volume is greater than the second volume.

18. A method for providing a multi-lumen intravenous tubing system, the method comprising:
providing a manifold comprising a first fluid flow path that extends from a first inlet to an outlet of the manifold, a second fluid flow path that extends from a second inlet to the outlet of the manifold; and
providing a multi-lumen tubing comprising a first end coupled to the outlet of the manifold such that a primary lumen of the multi-lumen tubing is fluidly coupled with only the first fluid flow path, and a secondary lumen of the multi-lumen tubing is fluidly coupled with only the second fluid flow path.

19. The method of claim 18, wherein providing a manifold further comprises providing a tertiary fluid flow path between the first fluid flow path to the second fluid flow path, such that at least a portion of a fluid can move from the first fluid flow path to the second fluid flow path.

20. The method of claim 19, further comprising providing a valve along the tertiary fluid flow path to selectively permit or resist movement of the fluid between the first fluid flow path and the second fluid flow path.
